(19) United States
(12) Patent Application Publication
(10) Pub. No.: US 2022/0249455 A1
(43) Pub. Date: Aug. 11, 2022

(54) ENTERIC PROTON PUMP INHIBITOR SOFTGEL CAPSULE

(71) Applicant: R.P. Scherer Technologies, LLC, Carson City, NV (US)

(72) Inventors: Humera Ahmad, Endeavour Hills (AU); Jing Lin, Keysborough (AU); Patrick McNamara, Huntingdale (AU)

(21) Appl. No.: 17/627,273

(22) PCT Filed: Jun. 29, 2020
(86) PCT No.: PCT/US20/40068

§ 371 Date: Jan. 14, 2022

Related U.S. Application Data
(60) Provisional application No. 62/874,157, filed on Jul. 15, 2019.

(51) Int. Cl. A61K 31/4439; A61K 9/48

(52) U.S. Cl. CPC A61K 31/4439; A61K 9/485; A61K 9/4858; A61K 9/4816

(57) ABSTRACT

Disclosed is an enteric softgel capsule composition comprising (a) a fill material comprising a proton pump inhibitor, and (b) an enteric shell composition. The enteric softgel composition being stable and exhibiting a target dissolution profile and softgel capsule rupture characteristics.

### RELATED APPLICATIONS

The present application claims priority to U.S. Provisional Patent Application No. 62/874,157, filed on Jul. 15, 2019, which is herein incorporated by reference in its entirety.

### FIELD OF THE INVENTION

The present invention relates to enteric softgel capsules, and specifically to enteric softgel capsules comprising a proton pump inhibitor and methods of preparation thereof.

### BACKGROUND OF THE INVENTION

Soft capsules, in particular, soft gelatin capsules (or softgel capsules), provide a dosage form which is more readily accepted by patients, since the capsules are easy to swallow and need not be flavored in order to mask any unpleasant taste of the active agent. Softgel encapsulation of drugs further provides the potential to improve the bioavailability of the pharmaceutical agents. For example, active ingredients may be rapidly released in liquid form as soon as the gelatin shell ruptures.

Enteric dosage forms are designed to protect the contents of the dosage form from gastric conditions and are particularly beneficial for delivery of active ingredients that may cause stomach irritation or are sensitive to the acidic environment of the stomach. There have been ongoing efforts to make the benefits associated with soft gelatin capsules available to active pharmaceutical ingredients that favor administration via enteric formulations.

### OBJECTS AND SUMMARY OF THE INVENTION

The present invention is directed to enteric softgel capsules comprising a proton pump inhibitor. The enteric softgel capsules comprises (a) a fill material comprising a proton pump inhibitor and (b) an enteric shell composition.

The fill material in the enteric softgel capsules disclosed herein may comprise one or more of proton pump inhibitor (e.g., esomeprazole), triglycerides, glyceryl monostearate, antioxidant, surfactant and/or dispersing agent, alkalizing agent, and/or wetting agent.

The enteric shell composition in the enteric softgel capsules disclosed herein may comprise one or more of carrageenan, gelatin, plasticizer, and/or solvent.

The enteric shell composition may have a pH ranging from about 7.0 to about 9.0.

The enteric softgel capsules disclosed herein may be free or substantially free of conventional enteric polymer materials.

The enteric softgel capsules disclosed herein may be free or substantially free of an enteric coating (i.e., in addition to the enteric shell composition).

The enteric softgel capsules disclosed herein may exhibit a dissolution profile wherein about 60% or more of the proton pump is dissolved in about 30 minutes at pH of about 6.8 during a buffer stage when the fill material is tested using UPLC and pursuant to USP41-NF36.

The enteric softgel capsules disclosed herein may not rupture for at least one hour when the softgel capsule is in 0.1N HCl at about 37±2° C.

The enteric softgel capsules disclosed herein may exhibit a stability wherein about 90% w/w or more of the proton pump inhibitor remains in the enteric softgel capsule after being subjected to a stability test performed under stressed conditions at a temperature of about 25° C. and 60% relative humidity for about 2 weeks.

The present invention is also directed to a process of making any of the enteric softgel capsules described herein by preparing the fill material and encapsulating the fill material with the enteric shell composition.

The present invention is also directed to a method of treating a condition treatable by a proton pump inhibitor (e.g., gastroesophageal reflux disease (GERD)) by administering any of the enteric softgel capsules described herein to a patient in need thereof in accordance with a treatment regimen.

### Definitions

As used herein, the terms “active agent” “pharmaceutically active ingredient” refer to any material that is intended to produce a therapeutic, prophylactic, or other intended effect and is used in the diagnosis, cure, mitigation, treatment or prevention of a condition, whether or not approved by a government agency for that purpose. These terms with respect to specific agents include all pharmaceutically active agents, all pharmaceutically acceptable salts thereof, stereoisomers, crystalline forms, co-crystals, ether, esters, hydrates, solvates, and mixtures thereof, where the form is pharmaceutically active.

The terms “treatment of” and “treating” include the administration of an active agent(s) with the intent to lessen the severity of or prevent a condition.

The terms “prevention of” and “preventing” include the avoidance of the onset of a condition.

The term “condition” or “conditions” refers to those medical conditions, such as gastroesophageal reflux disease (GERD), that can be treated, mitigated or prevented by administration to a subject of an effective amount of an active agent. Exemplary non-limiting conditions that may benefit from enteric softgel capsules may include, without limitation, conditions that may be treated with proton pump inhibitors.

The dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

An “effective amount” or a “therapeutically effective amount” refers to the amount of an active ingredient in a pharmaceutical composition that is sufficient to produce a beneficial or desired effect at a level that is readily detectable by a method commonly used for detection of such an effect. In some embodiments, such an effect results in a change of at least 10% from the value of a basal level where the active ingredient is not administered. In other embodiments, the change is at least 20%, 50%, 80%, or an even higher percentage from the basal level. As will be described below, the effective amount of an active ingredient may vary from subject to subject, depending on age, general condition of the subject, the severity of the condition being treated, and the particular active agent administered and the like. An appropriate “effective” amount in any individual case may be determined by one of ordinary skill in the art by reference to the pertinent texts and literature and/or by using routine experimentation.

As used herein, “shell” or “shell composition”, refers to the shell of a softgel capsule which encapsulates a fill material.

As used herein, “fill material” or “fill,” refers to the composition that is encapsulated by the enteric capsule shell composition and contains at least one pharmaceutically active ingredient.

As used herein, the term “enteric” is used to refer to the dissolution or disintegration resistant property of a substance such that dissolution or disintegration does not occur in a gastric environment. For example, the embodiments described herein include an enteric shell composition that dissolves in biological, artificial or simulated intestinal fluid rather than in biological, artificial or simulated gastric fluid.

As used herein, “enteric capsules” or “enteric softgel capsules” refer to capsules which have enteric properties once the fill material is encapsulated in the shell, and the capsules are dried. No further processing steps are required.

As used herein, a “patient” refers to a subject, particularly a human (but could also encompass a non-human), who has presented a clinical manifestation of a particular symptom or symptoms suggesting the need for treatment, who is treated prophylactically for a condition, or who has been diagnosed with a condition to be treated.

The term “subject” encompasses the definition of the term “patient” and does not exclude individuals who are otherwise healthy.

As used herein, the singular forms “a,” “an,” and “the” include plural references unless the context clearly indicates otherwise. Thus, for example, reference to “a proton pump inhibitor” includes a single proton pump inhibitor as well as a mixture of two or more different proton pump inhibitors; and reference to an “antioxidant” includes a single antioxidant as well as a mixture of two or more different antioxidants, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### DETAILED DESCRIPTION

The present invention advances the state of the art by developing enteric oral dosage forms, in particular, enteric softgel capsules for a proton pump inhibitor. This enteric softgel capsule achieves the advantages associated with the conventional enteric dosage forms without the need to add a conventional enteric polymer in the capsule shell. In certain embodiments, the enteric softgel capsules described herein achieves the advantages associated with the conventional enteric dosage forms while being free of an enteric coating. The enteric softgel capsules of the present invention do not dissolve in a gastric environment of the stomach, but rather dissolve in the intestines. Such mechanism is beneficial for delivery of proton pump inhibitors that may cause stomach irritation or are sensitive to the acidic environment of the stomach.

According to a first embodiment, an enteric softgel capsule comprises (a) a fill material comprising a proton pump inhibitor and (b) an enteric shell composition comprising carrageenan, wherein about 60% or more of the proton pump is dissolved in up to about 30 minutes at pH of about 6.8 during a buffer stage when the fill material is tested using UPLC and pursuant to USP41-NF36.

According to a second embodiment, an enteric softgel capsule comprises (a) a fill material comprising a proton pump inhibitor and (b) an enteric shell composition having a pH of about 7.0 to about 9.0, the enteric shell composition comprising carrageenan, wherein the enteric shell does not rupture for at least one hour when the softgel capsule is in 0.1N HCl at about 37±2° C.

According to a third embodiment, an enteric softgel capsule comprises (a) a fill material comprising a proton pump inhibitor and (b) an enteric shell composition comprising carrageenan, wherein about 90% w/w or more of the proton pump inhibitor remains in the enteric softgel capsule after being subjected to a stability test performed under stressed conditions at a temperature of about 25° C. and 60% relative humidity for about 2 weeks.

According to a fourth embodiment, an enteric softgel capsule comprises (a) a fill material comprising a proton pump inhibitor and triglycerides, and (b) an enteric shell composition, wherein about 60% or more of the proton pump is dissolved in up to about 30 minutes at pH of about 6.8 during a buffer stage when the fill material is tested using UPLC and pursuant to USP41-NF36.

According to a fifth embodiment, an enteric softgel capsule comprises (a) a fill material comprising a proton pump inhibitor and triglycerides and (b) an enteric shell composition having a pH of about 7.0 to about 9.0, wherein the enteric shell does not rupture for at least one hour when the softgel capsule is in 0.1N HCl at about 37±2° C.

According to a sixth embodiment, an enteric softgel capsule comprises (a) a fill material comprising a proton pump inhibitor and triglycerides, and (b) an enteric shell composition, wherein about 90% w/w or more of the proton pump inhibitor remains in the enteric softgel capsule after being subjected to a stability test performed under stressed conditions at a temperature of about 25° C. and 60% relative humidity for about 2 weeks.

According to a seventh embodiment, an enteric softgel capsule comprises (a) a fill material comprising a proton pump inhibitor, about 60% w/w to about 90% w/w of a suspension system, about 0.1% w/w to about 5% w/w of an antioxidant, about 0.5% w/w to about 5% w/w of a surfactant and/or dispersing agent, and about 0.5% w/w to about 10% w/w of an alkalizing agent, based on total weight of the fill material; and (b) an enteric shell composition, wherein the enteric softgel capsule is free of conventional enteric polymers, and wherein about 60% or more of the proton pump inhibitor is dissolved in up to about 30 minutes at pH of about 6.8 during a buffer stage when the fill material is tested using UPLC and pursuant to USP41-NF36.

According to an eighth embodiment, an enteric softgel capsule comprises (a) a fill material comprising a proton pump inhibitor, about 60% w/w to about 90% w/w of a suspension system, about 0.1% w/w to about 5% w/w of an antioxidant, about 0.5% w/w to about 5% w/w of a surfactant and/or dispersing agent, and about 0.5% w/w to about 10% w/w of an alkalizing agent, based on total weight of the fill material; and (b) an enteric shell composition, wherein the enteric softgel capsule is free of conventional enteric polymers, and wherein about 90% w/w or more of the proton pump inhibitor remains in the enteric softgel capsule after being subjected to a stability test performed under stressed conditions at a temperature of about 25° C. and 60% relative humidity for about 2 weeks.

According to a ninth embodiment, an enteric softgel capsule comprises (a) a fill material comprising a proton pump inhibitor, about 60% w/w to about 90% w/w of a suspension system, about 0.1% w/w to about 5% w/w of an antioxidant, about 0.5% w/w to about 5% w/w of a surfactant and/or dispersing agent, and about 0.5% w/w to about 10% w/w of an alkalizing agent, based on total weight of the fill material; and (b) an enteric shell composition, wherein the enteric softgel capsule is free of conventional enteric polymers, wherein enteric shell does not rupture for at least one hour when the softgel capsule is in 0.1N HCl at about 37±2° C.

According to a tenth embodiment, a fill material comprising a proton pump inhibitor, about 60% w/w to about 90% w/w of a suspension system, about 0.1% w/w to about 5% w/w of an antioxidant, about 0.5% w/w to about 5% w/w of a surfactant and/or dispersing agent, and about 0.5% w/w to about 10% w/w of an alkalizing agent, based on total weight of the fill material; and wherein about 60% or more of the proton pump inhibitor is dissolved in up to about 30 minutes at pH of about 6.8 during a buffer stage when the fill material is tested using UPLC and pursuant to USP41-NF36.

Enteric softgel capsules described herein may comprise about 10 mg to about 50 mg, about 15 mg to about 45 mg, about 18 mg to about 22 mg, about 38 mg to about 42 mg, about 20 mg, or about 40 mg of the proton pump inhibitor.

The proton pump inhibitor in the enteric softgel capsules disclosed herein may be esomeprazole or a pharmaceutically acceptable salt thereof, such as, without limitations, esomeprazole magnesium dihydrate. In one embodiment, the proton pump inhibitor is esomeprazole magnesium dehydrate having a molecular formula C₃₄H₃₆MgN₆O₆S₂.2H₂O with a molecular weight of 749.15 g/mol (chemical name: 5-methoxy-2-[(S)-[4-methoxy-3,5-dimethyl-2-pyridyl)methyl]sulfinyl]benzimidazole, magnesium salt (2:1), dehydrate; CAS #217087-10-0) and the following chemical structure:

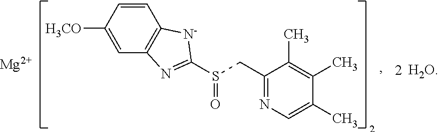

Esomeprazole magnesium is the magnesium salt of esomeprazole, the S-isomer of omeprazole, with gastric proton pump inhibitor activity. In the acidic compartment of parietal cells, esomeprazole is protonated and converted into the active achiral sulphenamide. The active sulphenamide forms one or more covalent disulfide bonds with the proton pump hydrogen-potassium adenosine triphosphatase (H+/K+ ATPase), thereby inhibiting its activity and the parietal cell secretion of H+ ions into the gastric lumen, the final step in gastric acid production. H+/K+ ATPase is an integral membrane protein of the gastric parietal cell. In one embodiment, the amount of esomeprazole magnesium dihydrate is 21.7 mg to provide 20 mg of esomeprazole.

In certain embodiments, enteric softgel capsules disclosed herein may further comprise further active ingredients in addition to the proton pump inhibitor. Further pharmaceutically active ingredient may include, without limitation, analgesics and anti-inflammatory agents, antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, oral vaccines, proteins, peptides and recombinant drugs, sex hormones and contraceptives, spermicides, stimulants, anticancer agents, and combinations thereof.

In some embodiments, the further active pharmaceutical ingredient may be selected, without limitations, from the group consisting of dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

In some embodiments, further active pharmaceutical ingredients may include lipids selected, without limitations, from the group consisting of almond oil, argan oil, avocado oil, borage seed oil, canola oil, cashew oil, castor oil, hydrogenated castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, hydrogenated vegetable oil, walnut oil, and watermelon seed oil. Other oil and fats may include, but not be limited to, fish oil (omega-3), krill oil, animal or vegetable fats, e.g., in their hydrogenated form, free fatty acids and mono-, di-, and tri-glycerides with C8-, C10-, C12-, C14-, C16-, C18-, C20- and C22-fatty acids, and combinations thereof.

According to certain embodiments, further active pharmaceutical ingredients may include lipid-lowering agents including, but not limited to, statins (e.g., lovastatin, simvastatin, pravastatin, fluvastatin, atorvastatin, rosuvastatin, and pitavastatin), fibrates (e.g, clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical as further active pharmaceutical ingredients may include, but not be limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sequioxide, glandular products, glucosamine HCl, glucosamine sulfate, hydroxyl methyl butyrate, immunoglobulin, lactic acid, L-Camitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplements as further active pharmaceutical ingredients may include vitamins, minerals, fiber, fatty acids, amino acids, herbal supplements or a combination thereof.

Suitable vitamin active as further active pharmaceutical ingredients may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Suitable herbal supplement as further active pharmaceutical ingredients may include, but are not limited to, the following: arnica, bilberry, black cohosh, cat's claw, chamomile, echinacea, evening primrose oil, fenugreek, flaxseed, feverfew, garlic, ginger root, ginko biloba, ginseng, goldenrod, hawthorn, kava-kava, licorice, milk thistle, psyllium, rauowolfia, senna, soybean, St. John's wort, saw palmetto, turmeric, valerian.

Minerals as further active pharmaceutical ingredients may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Examples of other possible further active pharmaceutical ingredients include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib, Cox-2 inhibitors, diclofenac, benoxaprofen, flurbiprofen, fenoprofen, flubufen, indoprofen, piroprofen, carprofen, oxaprozin, pramoprofen, muroprofen, trioxaprofen, suprofen, aminoprofen, fluprofen, bucloxic acid, indomethacin, sulindac, zomepirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, faislamine, fenbufen, fenoprofen, flurbiprofen, ibuprofen, indometacin, ketoprofen, ketorolac, lomoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin. pharmaceutically acceptable salts thereof and mixtures thereof) and acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The further active pharmaceutical ingredient may also be a benzodiazepine, barbiturate, stimulants, or mixtures thereof. The term “benzodiazepines” refers to a benzodiazepine and drugs that are derivatives of a benzodiazepine that are able to depress the central nervous system. Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, methylphenidate as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2, 4, 6-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

In addition to the proton pump inhibitor, the fill may further comprise suitable fill materials such as flavoring agents, sweetening agents, coloring agents and fillers or other pharmaceutically acceptable excipients or carriers. The term “pharmaceutically acceptable excipient or carrier” refers to any inert ingredient in a composition that may act, for example, to stabilize the active ingredient. A pharmaceutically acceptable excipient can include, but is not limited to, carbohydrates (such as glucose, sucrose, or dextrans), antioxidants (such as d-α-tocopherol, ascorbic acid, or glutathione), chelating agents, low-molecular weight proteins, high-molecular weight polymers, gel-forming agents or other stabilizers and additives. Other examples of a pharmaceutically acceptable carrier include wetting agents, emulsifying agents, surfactant and/or dispersing agents, alkalizing agents, or preservatives, which are particularly useful for preventing the growth or action of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, Pa., 17th ed. (1985).

In one embodiment, the fill material may comprise medium chain triglycerides. In one embodiment, the fill material may comprise glyceryl monostearate. In certain embodiments, the fill material may comprise a suspension system comprising medium chain triglycerides and glyceryl monostearate. The suspension system may be present in the fill material at about 60% w/w to about 90% w/w, about 65% w/w to about 85% w/w, or about 70% w/w to about 80% w/w, based on total weight of the fill material. The w/w ratio of the medium chain triglycerides to the glyceryl monostearate in the suspension system may be about 1:1 to about 50:1, about 5:1 to about 40:1, about 8:1 to about 30:1, or about 10:1 to about 25:1.

In certain embodiments, the fill material includes an antioxidant that is one or more of alpha-tocopherol, beta-tocopherol, gamma-tocopherol, delta-tocopherol, or a combination thereof. In one embodiment, the fill material may comprise an antioxidant that is d-α-tocopherol. The antioxidant may be present in the fill material at about 0.1% w/w to about 5% w/w, about 0.5% w/w to about 4% w/w, about 1% w/w to about 3% w/w, or about 1.5% w/w to about 2.5% w/w, based on total weight of the fill material.

In one embodiment, the fill material may comprise a surfactant and/or dispersing agent, such as polysorbate 80. The surfactant and/or dispersing agent may be present in the fill material at about 0.5% w/w to about 5% w/w, about 1% w/w to about 5% w/w, about 2% w/w to about 5% w/w, about 3% w/w to about 5% w/w, or about 4% w/w to about 5% w/w, based on total weight of the fill material.

In one embodiment, the fill material may comprise an alkalizing agent, such as magnesium oxide. The alkalizing agent may be present in the fill material at about 0.5% w/w to about 10% w/w, about 2% w/w to about 10% w/w, about 5% w/w to about 10% w/w, or about 7% w/w to about 9% w/w, based on total weight of the fill material.

In one embodiment, the fill material may comprise a wetting agent, such as lecithin. The wetting agent may be present in the fill material at from about 0.5 w/w to about 5% w/w, from about 1% w/w to about 3% w/w, or from about 1.5% w/w to about 2.5% w/w, based on total weight of the fill material.

In an embodiment, the fill material may comprise from about 70% w/w to about 80% w/w of a suspension system, from about 1.5% w/w to about 2.5% w/w of an antioxidant, from about 4% w/w to about 5 w/w of a surfactant and/or dispersing agent, and from about 7% w/w to about 9% w/w of an alkalizing agent.

Enteric softgel capsules disclosed herein may be free or substantially free of conventional enteric polymers.

As used herein, “conventional enteric polymers” refer to, but are not limited to, acrylic and methacrylic acid polymers, which may be available under the tradename EUDRAGIT® and other conventional acid insoluble polymers, e.g., methyl acrylate-methacrylic acid copolymers. Other conventional acid insoluble polymers include, without limitation, cellulose acetate succinate, cellulose acetate phthalate, cellulose acetate butyrate, hydroxypropyl methyl cellulose phthalate, hydroxypropyl methyl cellulose acetate succinate (hypromellose acetate succinate), polyvinyl acetate phthalate (PVAP), algenic acid salts such as sodium alginate and potassium alginate, stearic acid and shellac. In some embodiments, the enteric shell composition of the present invention does not include an acid insoluble polymer. In other words, the enteric shell composition and the enteric softgel capsule are “free or substantially free of conventional enteric polymers.”

As used herein, “free or substantially free,” refers to a composition that comprises less than about 1% w/w, less than about 0.5% w/w, less than about 0.25% w/w, less than about 0.1% w/w, less than about 0.05% w/w, less than about 0.01% w/w, or 0% w/w of said component in the composition, based on total weight of the shell composition.

In certain embodiment, the carrageenan in the enteric shell composition may be kappa-carrageenan, iota-carrageenan, lambda-carrageenan and mixtures thereof. According to one embodiment, the carrageenan is kappa-carrageenan. According to another embodiment, the carrageenan is iota-carrageenan. In an embodiment, the amount of the carrageenan in the enteric shell composition is about 2% w/w to about 10% w/w, from about 2% w/w to about 8% w/w, or from about 2% w/w to about 5% w/w, based on total weight of the enteric shell composition.

In one embodiment, the enteric shell composition comprises gelatin. The gelatin in the enteric shell composition may include, but is not limited to, Type A gelatin, Type B gelatin, a hide gelatin, a fish gelatin, porcine gelatin and/or a bone gelatin used alone or in combination. In an embodiment, the gelatin is Type A high bloom gelatin. In an embodiment, the gelatin is Type B high bloom gelatin. In one embodiment, the gelatin is a 250 bloom gelatin. In another embodiment, there is only one type of gelatin. In yet another embodiment, the gelatin is a combination of at least two types of gelatins. In an embodiment, the amount of gelatin in the enteric shell composition is about 1% w/w to about 50% w/w, about 5% w/w to about 40% w/w, or about 10% w/w to about 30% w/w, based on total weight of the enteric shell composition.

In an embodiment, the enteric shell composition comprises a plasticizer. The plasticizer in the enteric shell composition may include glycerol, glycerin, sorbitol or a mixture thereof. Other suitable plasticizers may include, but not be limited to, sugar alcohol plasticizer such as isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof. In an embodiment, the amount of plasticizer in the enteric shell composition is 1% w/w to about 55% w/w, about 5% w/w to about 45% w/w, or about 10% w/w to about 35% w/w, based on total weight of the enteric shell composition.

In an embodiment, the enteric shell composition comprises a solvent. The solvent in the enteric shell composition may be or include water. In an embodiment, the amount of solvent in the capsule shell is from about 5% w/w to about 60% w/w, about 10% w/w to about 55% w/w, or about 20% w/w to about 50% w/w, based on total weight of the enteric shell composition.

In an embodiment, the enteric shell composition also includes a buffer and/or an alkalizing agent. A suitable buffer and/or alkalizing agent includes, but is not limited to, ammonium hydroxide, sodium hydroxide, sodium carbonate, sodium citrate, trisodium phosphate and/or disodium phosphate. In one embodiment, the buffer is disodium phosphate. In an embodiment, the amount of the buffer in the enteric shell composition is about 0.1% w/w to about 3% w/w, about 0.5% w/w to about 1% w/w, or about 0.5% w/w to about 0.9% w/w.

In an embodiment, the enteric shell composition may optionally comprise additional agents such as coloring agents, flavorings agents, sweetening agents, fillers, antioxidants, diluents or other pharmaceutically acceptable excipients or additives such as synthetic dyes and mineral oxides.

Exemplary suitable coloring agents may include, but are not limited to, colors such as e.g., white, black, yellow, blue, green, pink, red, orange, violet, indigo, and brown. In specific embodiments, the color of the dosage form can indicate the contents (e.g., one or more active ingredients) contained therein.

Exemplary suitable flavoring agents may include, but are not limited to, “flavor extract” obtained by extracting a part of a raw material, e.g., animal or plant material, often by using a solvent such as ethanol or water; natural essences obtained by extracting essential oils from the blossoms, fruit, roots, etc., or from the whole plants.

Additional exemplary flavoring agents that may be in the dosage form may include, but are not limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

Exemplary sweetening agents may include, but are not limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

In certain embodiments, about 70% or more, about 75% or more, about 80% or more, about 85% or more, or about 90% or more of the proton pump inhibitor is dissolved in up to about 45 minutes, up to about 30 minutes, up to about 20 minutes, or up to about 10 minutes (or in any single value or sub-range therein) at pH of about 6.8 during a buffer stage when the fill material is tested using UPLC and pursuant to USP41-NF36.

In certain embodiments, about 90% w/w or more, about 95% w/w or more, about 98% w/w or more, about 99% w/w or more, or about 99.5% w/w or more, or about 99.8% w/w or more of the proton pump inhibitor remains in the enteric softgel capsule after being subjected to a stability test performed under stressed conditions at a temperature of about 25° C. and 60% relative humidity for about 2 weeks.

In certain embodiments, about 90% w/w or more, about 95% w/w or more, wherein about 98% w/w or more, about 99% w/w or more, or about 99.5% w/w or more, or about 99.8% w/w or more of the proton pump inhibitor remains in the enteric softgel capsule after being subjected to a stability test performed under stressed conditions at a temperature of about 40° C. and 75% relative humidity for about 2 weeks.

In certain embodiments, about 90% w/w or more, about 95% w/w or more, wherein about 98% w/w or more, about 99% w/w or more, or about 99.5% w/w or more, or about 99.8% w/w or more of the proton pump inhibitor remains in the enteric softgel capsule after being subjected to a stability test performed under stressed conditions at a temperature of about 25° C. and 60% relative humidity for about 4 weeks.

In certain embodiments, about 90% w/w or more, about 95% w/w or more, wherein about 98% w/w or more, about 99% w/w or more, or about 99.5% w/w or more, or about 99.8% w/w or more of the proton pump inhibitor remains in the enteric softgel capsule after being subjected to a stability test performed under stressed conditions at a temperature of about 30° C. and 75% relative humidity for about 4 weeks.

Certain proton pump inhibitors, such as esomeprazole, are unstable in acidic environment. Therefore, an enteric softgel capsule that could pass the stomach acid and disintegrate in the intestines to release the proton pump inhibitor is advantageous.

The enteric softgel capsule according to an embodiment may remain intact for at least about one hour, at least about two hours, at least about three hours, at least about four hours, at least about five hours, or longer than about 1-5 hours in acidic medium and may disintegrate in intestinal fluid in about 120 minutes or less, or about 100 minutes or less, or about 80 minutes or less, or about 60 minutes or less, or about 45 minutes or less, or about 30 minutes or less, or about 10 minutes or less, or about 5 minutes or less. In certain embodiments, the enteric shell does not rupture for at least about one hour, at least about two hours, at least about three hours, at least about four hours, or at least about five hours when the softgel capsule is in 0.1N HCl at about 37±2° C.

The disintegration may be measured using the in Line BP/USP method. For example, using USP <701> Disintegration test, the softgel capsules can be disintegrated in a basket-rack assembly. The basket-rack assembly can include six-open ended transparent tubes, each 77.5±2.5 mm long and having an inside diameter of 20.7 to 23 mm and a wall 1.0 to 2.8 mm thick; the tubes are held in a vertical position by two plates, each 88 to 92 mm in diameter and 5 to 8.5 mm in thickness, with six holes, each 22 to 26 mm in diameter, equidistant from the center of the plate and equally spaced from one another. Attached to the under surface of the lower plate is a woven stainless steel weave with 1.8- to 2.2-mm apertures and with a wire diameter of 0.57 to 0.66 mm. The parts of the apparatus are assembled and rigidly held by means of three bolts passing through the two plates. A suitable means is provided to suspend the basket-rack assembly from the raising and lowering device using a point on its axis. The use of disks is permitted only where specified or allowed in the monograph. Additionally, a removable wire cloth having the aforementioned weave and diameter specifications is attached to the surface of the upper plate of the basket-rack assembly.

One (1) dosage unit is placed in each of six tubes of the basket of a basket-rack assembly and, if necessary, a disk is added. Each enteric softgel capsule is immersed in 0.1 N HCl, which is maintained at a temperature of 37° C.±2° C. After 120 minutes, or 100 minutes, or 80 minutes, or 60 minutes, or 45 minutes, or 30 minutes, or 10 minutes, or 5 minutes, the basket is lifted from the fluid, and the enteric softgel capsules are observed to see if they show no evidence of disintegration. If 1 or 2 enteric softgel capsules disintegrate, the test is repeated on 12 additional enteric softgel capsules. The requirement is met if 16 or more of the total of 18 enteric softgel capsules tested show no evidence of disintegration.

Enteric softgel capsules comprising esomeprazole or a pharmaceutically acceptable salt thereof as the proton pump inhibitor may have an alkaline enteric shell composition due to esomeprazole being sensitive to acidic conditions. Thus, in some embodiments, the shell composition has a pH of about 7.0 to about 9.0, about 7.0 to about 8.0, about 8.0 to about 9.0, about 7.5 to about 8.5, about 7.5 to about 9.0, about 7 to about 8.5, about 7.0 to about 7.5, about 7.5 to about 8.0, about 8.0 to about 8.5, about 8.5 to about 9.0, or about 8.6. In an embodiment, the shell composition may contain sodium hydroxide to maintain the shell pH.

Encapsulation of the fill material can be accomplished using any conventional manner. As an example, a rotary die encapsulation may be used. In embodiments, the encapsulation process can use equipment and processes for vegetarian capsules due to the relatively high melting point of the gel in the enteric softgel capsule.

According to an embodiment, any of the enteric softgel capsules disclosed herein may be prepared by a process comprising the steps of: (a) preparing the fill material; and (b) encapsulating the fill material of step (a) in an enteric shell composition. The encapsulation process according to step (a) may further comprise one or more of the following sub-steps: (a)(1) adding medium chain triglycerides and glyceryl monostearate to a melter to form a suspension system, (a)(2) mixing a suspension system at a temperature of about 60° C. to about 70° C. under vacuum, (a)(3) adding a wetting agent, an antioxidant, and a surfactant and/or dispersing agent to a suspension system at a temperature of about 35° C. to about 45° C., and/or (a)(4) adding an alkalizing agent and a proton pump inhibitor to a suspension system at a temperature of about 20° C. to about 30° C. The encapsulation process according to step (b) may further comprise one or more of the following sub-steps: (b)(1) adding a solvent, a plasticizer, and gelatin to a melter to form a mixture, (b)(2) mixing a mixture of a solvent, a plasticizer, and gelatin at a temperature of about 70° C. to about 80° C. under vacuum, (b)(3) heating a mixture of a solvent, a plasticizer, and gelatin to a temperature of about 80° C. to about 90° C., and/or (b)(4) combining a mixture of a solvent, a plasticizer, and gelatin with a carrageenan-based premix.

The enteric quality and acid resistance of the enteric softgel capsules disclosed herein rely, among other factors, on strong capsule seal which is commonly known as the weakest part of a softgel capsule. Strong seal can be achieved by a combination of strong gel, skillful design of the die tooling and control of the process parameters during encapsulation.

### EXAMPLES

The following prophetic examples are set forth to assist in understanding the invention and should not be construed as specifically limiting the invention described and claimed herein. Such variations of the invention, including the substitution of any or all equivalents now known or later developed, which would be within the purview of those skilled in the art, and changes in formulation or minor changes in therapeutic design, are to be considered to fall within the scope of the invention incorporated herein.

Many trials were conducted to determine suitable excipient base to achieve a fill formulation that meets the following requirement: physically and chemically stable suspension, smallest capsule size, fast dissolution, suitable viscosity for encapsulation.

Various carrier liquids, suspending agents and antioxidants were screened. Fill viscosity was measured using rheometer and dissolution performance was determined by visual observation and chemical assay. The fill samples were stressed at various temperature and humidity to check for physical stability of the suspension.

Initial trials used a proprietary material called Geloil SC which consists of hydrogenated soya bean oil, glyceryl distearate and polyglyceryl-3 dioleate. The advantage of using Geloil SC is that it worked as a carrier and suspending agent. Geloil SC also has good dispersion properties which is advantageous for this product. See table 1 for the initial formulation.

TABLE 1
Initial Fill Formulation

| Ingredients | mg/caps | % w/w |
|---|---|---|
| Esomeprazole magnesium | 21.7 | 9.32 |
| Geloil SC | 189.5 | 80.30 |
| Ascorbyl Palmitate | 2.5 | 1.06 |
| Magnesium Oxide | 22 | 9.32 |
| Total | 236 | 100 |

Additional excipients that were investigated were soya bean oil and medium chain triglycerides as the potential liquid carriers; yellow beeswax, hydrogenated vegetable oil and glyceryl monostearate as the suspending agents; lecithin as a wetting agent, and polysorbate 80 as a surfactant/dispersing agent.

Soya bean oil, yellow beeswax and hydrogenated vegetable oil were grouped together as one suspending system. Medium chain triglyceride and glyceryl monostearate were grouped together as another suspending system.

Examples of the excipient systems trialed are referred to in tables 2 and 3.

TABLE 2
Excipient Base Using Soya Bean Oil

| Ingredients | % w/w |
|---|---|
| Soya bean oil | 55 |
| Yellow beeswax | 10 |
| Hydrogenated vegetable oil | 25 |
| Lecithin | 5 |
| Polysorbate 80 | 5 |
| Total | 100 |

TABLE 3
Excipient Base Using Medium Chain Triglycerides

| Ingredients | % w/w |
|---|---|
| Medium chain triglycerides (MCT) | 75 |
| Glyceryl monostearate | 10 |
| Lecithin | 5 |
| Polysorbate 80 | 10 |
| Total | 100 |

These two base formulations of Tables 2 and 3 were tested for their dispersibility in water to ensure that the fill disperses sufficiently during dissolution testing. Both base formulations of Tables 2 and 3 showed poor dispersion compared to Geloil SC.

Variation trials on the excipient base formulations of table 2 were conducted altering the amounts of all the ingredients. The one which was selected for trials with Esomeprazole Magnesium is shown in Table 4.

TABLE 4
Excipient Base Used for Trials with Esomeprazole Magnesium

| Ingredients | % w/w |
|---|---|
| Soya bean oil | 68 |
| Yellow beeswax | 10 |
| Hydrogenated vegetable oil | 15 |
| Lecithin | 5 |
| Polysorbate 80 | 2 |
| Total | 100 |

The first esomeprazole magnesium trial used the excipient base formulation of Table 4 with the addition of ascorbyl palmitate antioxidant and magnesium oxide alkalizing agent as shown in Table 5.

TABLE 5
First Trial with Esomeprazole Magnesium

| Ingredients | mg/capsule |
|---|---|
| Excipient mix of Table 4 | 189.5 |
| Ascorbyl Palmitate | 2.5 |
| Magnesium Oxide - Heavy | 22 |
| Esomeprazole Mg dihydrate | 22 |
| Total | 236 |

The trial in Table 5 produced a stable suspension however in under 24 hours brown specks started to appear when stored at room temperature, indicating oxidation of esomeprazole.

The decision to switch from soya bean oil to medium chain triglycerides (MCT) was made due to MCT having a lower peroxide limit which would help reduce the oxidation observed with the trial in Table 5.

TABLE 6
Trial with Medium Chain Triglycerides
Base with Esomeprazole Magnesium

| Ingredients | mg/capsule |
|---|---|
| Medium chain triglycerides | 160 |
| Glyceryl monostearate | 25 |
| Ascorbyl Palmitate | 2.5 |
| Lecithin | 9.2 |
| Polysorbate 80 | 5 |
| Magnesium Oxide - Heavy | 22 |
| Esomeprazole Mg dihydrate | 22 |
| Total | 245.7 |

The formulation in Table 6 showed some improvement but still brown specks were seen throughout the fill. It was noted that the ascorbyl palmitate was not dispersing evenly through the fill material hence not providing adequate antioxidant properties to prevent the Esomeprazole Magnesium from oxidizing.

D-a-Tocopherol was selected as the replacement for ascorbyl palmitate as it is more readily oil soluble anti-oxidant that is compatible with the other excipients used in the formulation. See Table 7 for the formulation with d-a-Tocopherol.

TABLE 7
trial with d-a-Tocopherol

| Ingredients | mg/capsule |
|---|---|
| Medium chain triglycerides | 160 |
| Glyceryl monostearate | 15 |
| d-a-Tocopherol | 2.5 |
| Lecithin | 9.2 |
| Polysorbate 80 | 5 |
| Magnesium Oxide - Heavy | 22 |
| Esomeprazole Mg dihydrate | 22 |
| Total | 235.7 |

The formulation in Table 7 showed good physical stability and there were no brown specks observed when the fill was stored at room temperature. This sample was then tested for visual dissolution properties. The sample showed a good dispersion in water however there was several large globules of undispersed fill material, which contained part of the Esomeprazole Magnesium solids.

A finer dispersion of the fill material was required to meet dissolution testing as per USP41-NF36, Esomeprazole Magnesium Delayed-Release Capsule monograph, of not less than 75% (Q) of the labelled amount of Esomeprazole being dissolved in 30 minutes at pH 6.8 during the buffer stage. The shell for the soft capsule would need to not rupture during the first 2 hours when the capsule is in 0.1 N HCl.

Further trials were conducted. The fill formulation in Table 8 was selected for chemical dissolution testing following the parameters set out in the monograph in USP41-NF36 and the analysis using UPLC1.

TABLE 8
Fill Formulation 1 Tested for Dissolution and Analysis on UPLC

| Ingredients | mg/capsule |
|---|---|
| Medium chain triglycerides | 180 |
| Glyceryl monostearate | 15 |
| d-a-Tocopherol | 5 |
| Lecithin | 9.2 |
| Polysorbate 80 | 5 |
| Magnesium Oxide - Heavy | 22 |
| Esomeprazole Mg dihydrate | 21.7 |
| Total | 257.9 |

The sample tested from the formulation in Table 8 showed good dispersion and the dissolution results. Approximately 60% of Esomeprazole Magnesium was released. This showed some improvement was required to achieve a target of 90% release. A few more trial samples were made, altering different excipients to improve its dispersion in the dissolution medium. The formulation tested for dissolution had a reduction in glyceryl monostearate and lecithin. The formula is shown in table 9.

TABLE 9
Fill Formulation 2 Tested for Dissolution and Analysis on UPLC

| Ingredients | mg/capsule |
|---|---|
| Medium chain triglycerides | 180 |
| Glyceryl monostearate | 10 |
| d-a-Tocopherol | 5 |
| Lecithin | 5 |
| Polysorbate 80 | 5 |
| Magnesium Oxide - Heavy | 22 |
| Esomeprazole Mg dihydrate | 21.7 |
| Total | 248.7 |

The dissolution from Table 9 formula has increased to 78% release as compared to 60% from the formula in Table 8. Still higher dissolution was targeted so another series of trials were conducted mainly concentrating on the effect in increasing the amount of Polysorbate 80 in the formulation. The formulations in Tables 10 and 11 were tested for dissolution.

TABLE 10
Fill Formulation 3 Tested for Dissolution and Analysis on UPLC

| Ingredients | mg/capsule |
|---|---|
| Medium chain triglycerides | 180 |
| Glyceryl monostearate | 10 |
| d-a-Tocopherol | 5 |
| Lecithin | 5 |
| Polysorbate 80 | 7 |
| Magnesium Oxide - Heavy | 22 |
| Esomeprazole Mg dihydrate | 21.7 |
| Total | 250.7 |

TABLE 11
Fill Formulation 4 Tested for Dissolution and Analysis on UPLC

| Ingredients | mg/capsule |
|---|---|
| Medium chain triglycerides | 180 |
| Glyceryl monostearate | 10 |
| d-a-Tocopherol | 5 |
| Lecithin | 5 |
| Polysorbate 80 | 9 |
| Magnesium Oxide - Heavy | 22 |
| Esomeprazole Mg dihydrate | 21.7 |
| Total | 252.7 |

The dissolution result based on Table 10 formula was 77% and the result for Table 11 was 83%. The results were promising and showed improvement with the addition of extra Polysorbate 80 allowing for a finer dispersion. The result was still short by 10% of Esomeprazole release to meet the dissolution target.

The two following trials tested increased the amount of Polysorbate 80 and medium chain triglycerides to improve dispersion. In the formulation presented in Table 13, the amount of magnesium oxide was reduced to assess if it had any impact on the dispersion and dissolution.

TABLE 12
Fill Formulation 5 Tested for Dissolution and Analysis on UPLC

| Ingredients | mg/capsule |
|---|---|
| Medium chain triglycerides | 200 |
| Glyceryl monostearate | 10 |
| d-a-Tocopherol | 5 |
| Lecithin | 5 |
| Polysorbate 80 | 12 |
| Magnesium Oxide - Heavy | 22 |
| Esomeprazole Mg dihydrate | 21.7 |
| Total | 275.7 |

TABLE 13
Fill Formulation 6 Tested for Dissolution and Analysis on UPLC

| Ingredients | mg/capsule |
|---|---|
| Medium chain triglycerides | 200 |
| Glyceryl monostearate | 10 |
| d-a-Tocopherol | 5 |
| Lecithin | 5 |
| Polysorbate 80 | 12 |
| Magnesium Oxide - Heavy | 5 |
| Esomeprazole Mg dihydrate | 21.7 |
| Total | 258.7 |

The results of dissolution for the sample based on Table 12 was 93% and the results for Table 14 was 89%. The result from the formula in Table 12 has achieved the target dissolution.

More samples were made based on the formula in Table 12 and stored in amber glass bottles, then stressed under controlled conditions to observe if the formulation was stable under higher temperatures and higher humidity. Refer to table 14 for results.

TABLE 15
Assay Results of Fill Formula 5 Samples
Stressed in Stability Chambers

| | 2 weeks | 4 weeks |
|---|---|---|
| | % Label Claim of | % Label Claim of |
| Sample Name | Esomeprazole | Esomeprazole |
| Placebo 5° C./AH | NA | NA |
| Placebo 25° C./60% RH | NA | NA |
| Placebo 40° C./75% RH | NA | NA |
| Sample 5° C./AH | 101.9 | 99.8 |
| Sample 25° C./60% RH | 106.5 | 99.9 |
| Sample 30° C./75% RH | NA | 101.3 |
| Sample 40° C./75% RH | 102.5 | 79.7 |

These results show that under the stress conditions the formulation is stable up to 30° C./75% RH for at least 4 weeks.

### Shell Formulation

A pouch was made using a gel formulation as per Table 15 and the fill material from the formulation in Table 12. The pouch was stressed at 30° C./AH for 28 days. The fill did not show visible signs of discoloration, which indicated the fill was stable.

TABLE 15
Alkaline Enteric Gel Formulation

| Soft Capsule Shell | g/kg gel |
|---|---|
| Gelatin | 264 |
| Glycerol | 250 |
| Carrageenan | 29 |
| Water-Purified | 461 |
| Titanium Dioxide | 1 |
| Quinoline Yellow CI47005 | 0.05 |
| Brilliant Blue FCF CI42090 | 0.3 |
| Polysorbate 80 | 0.0005 |
| Sodium Hydroxide | 2 |
| Silica-Colloidal Anhydrous | 0.05 |
| Total | 1007.4 |

### Fill Material Manufacturing Process According to an Embodiment

Step 1: Set vacuum on a vacuum mixing vessel at 70 kPa.

Step 2: Vacuum transfer in medium chain triglycerides and turn on agitator shear mixer and recirculation system.

Step 3: While stirring and recirculating, vacuum transfer in glyceryl monostearate and continue stirring until mix is uniform (about 5 minutes).

Step 4: While stirring and recirculating, start heating to reach a temperature between 60 to 70° C. and vacuum between 60-80 kPa. Continue stirring until all wax is melted and a clear solution is obtained (about 15 minutes) from when the temperature reaches 60° C.

Step 5: While stirring, cool the mix to 35° C. to 45° C.

Step 6: Transfer into the vessel lecithin, d-α-Tocopherol, and polysorbate 80.

Step 7: Continue stirring for 15 minutes and cool the mix to 20° C. to 30° C.

Step 8: In the tank, while stirring and maintaining set vacuum to 70 kPa, transfer into the vessel magnesium oxide—heavy and Esomeprazole Magnesium.

Step 9: Using the agitator, high shear mixer and recirculation, continue mixing under 70 kPa for 30 minutes until an even suspension is formed.

Step 10: Turn off shear mixer and while stirring with agitator and recirculation, cool the fill to 20° C. to 30° C.

Step 11: Turn off agitator and recirculation, keep vacuum at 60 kPa to 80 kPa and deaerate for at least 15 minutes.

Step 12: Return the tank to atmospheric pressure by gently pulsing nitrogen gas into the tank.

### Enteric Shell Composition Manufacturing Process According to an Embodiment

Step 1: Add the gelatin, purified water and 60% of the glycerine to vessel.

Step 2: Heat the vessel to 75° C. and mix under vacuum (60 to 80 kPa) and maintain at 70° C. to 80° C. for approximately 30 minutes. Then heat the vessel to 85° C.

Step 3: Whilst the vessel is reaching 85° C., premix the carrageenan with the remaining glycerine so that the carrageenan is just wetted with the glycerine.

Step 4: Once the vessel is at 85° C., transfer the premix of carrageenan and glycerine into the vessel, mix whilst maintaining the vessel at 85° C. and 60 to 80 kPa, and deaerate.

Step 5: Transfer the molten gel mass to stainless steel holding vessels.

Step 6: Add the shell colours and the sodium hydroxide as a 25% w/w solution in water to the molten gel mass and mix until fully dispersed and dissolved.

### Enteric Softgel Capsule Composition of Esomeprazole Magnesium Dihydrate

Esomeprazole enteric softgel capsule composition for oral administration is summarized in Table 16. The capsule may have an oval shape and an opaque blue colored shell. The esomeprazole enteric softgel formulation of Table 16 contains 20 mg of esomeprazole as a suspension fill. In this formulation, the drug substance is suspended in a liquid fill. Particle size is controlled by raw material specification that meets standard softgel requirement of less than 180 μm. The particle size physical characteristic is suitable for the softgel capsules.

TABLE 16
Esomeprazole Enteric Softgel Capsule Formulation

| Ingredients | mg/caps | Function |
|---|---|---|
| Esomeprazole magnesium | 1.7 | Active Pharmaceutical Ingredient |

Fill Excipients

| | | |
|---|---|---|
| Medium chain triglycerides | 200 | Carrier |
| Glyceryl monostearate | 10 | Suspending agent |
| Lecithin | 5 | Wetting agent/disintegrant |
| d-a-Tocopherol | 5 | Antioxidant |
| Polysorbate 80 | 12 | Surfactant |
| Magnesium oxide | 22 | Alkalizing agent |

Shell Excipients

| | |
|---|---|
| Gelatin | Shell material |
| Glycerol | Plasticizer |
| Carrageenan | Shell material |
| Titanium oxide | Colourant |
| Quinoline yellow CI47005 | Colourant |
| Brilliant blue FCF CI42090 | Colourant |
| Polysorbate 80 | Dispersant |
| Water - purified | Co-solvent |
| Sodium hydroxide | Alkalizing agent |
| Silica - colloidal anhydrous | Suspending agent |
| Total | |

### Packaging of the Drug Product

The product was to be packed in an opaque triplex blister strip comprising PVC/PE/PVDC, 250 μm/25 μm/90 gsm sealed with 20 μm hard tempered aluminum lidding foil. The blister strips were to be packed into cartons.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is simply intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. An enteric softgel capsule comprising:
(a) a fill material comprising a proton pump inhibitor; and
(b) an enteric shell composition,
wherein the fill material further comprises triglycerides or the enteric shell composition comprises carrageenan; and
wherein about 60% or more of the proton pump is dissolved in about 30 minutes at pH of about 6.8 during a buffer stage when the fill material is tested using UPLC and pursuant to USP41-NF36.

2. An enteric softgel capsule comprising:
(a) a fill material comprising a proton pump inhibitor; and
(b) an enteric shell composition having a pH of about 7.0 to about 9.0,
wherein the fill material further comprises triglycerides or the enteric shell composition comprises carrageenan; and
wherein the enteric shell does not rupture for at least one hour when the softgel capsule is in 0.1N HCl at about 37±2° C.

3. An enteric softgel capsule comprising:
(a) a fill material comprising a proton pump inhibitor; and
(b) an enteric shell composition,
wherein the fill material further comprises triglycerides or the enteric shell composition comprises carrageenan; and
wherein about 90% w/w or more of the proton pump inhibitor remains in the enteric softgel capsule after being subjected to a stability test performed under stressed conditions at a temperature of about 25° C. and 60% relative humidity for about 2 weeks.

4. The enteric softgel capsule of claim 1, wherein the proton pump inhibitor is esomeprazole or a pharmaceutically acceptable salt thereof.

5. The enteric softgel capsule of claim 1, wherein the proton pump inhibitor is esomeprazole magnesium dihydrate.

6. The enteric softgel capsule of claim 1, comprising from about 10 mg to about 50 mg of the proton pump inhibitor.

7.-10. (canceled)

11. The enteric softgel capsule of any of the preceding claims, wherein the fill material comprises a suspension system comprising medium chain triglycerides, glyceryl monostearate, or a combination thereof.

12. (canceled)

13. The enteric softgel capsule of claim 1, wherein the fill material comprises a suspension system comprising medium chain triglycerides and glyceryl monostearate, wherein the medium chain triglycerides to glyceryl monostearate w/w ratio is about 1:1 to about 50:1, about 5:1 to about 40:1, about 8:1 to about 30:1, or about 10:1 to about 25:1.

14. (canceled)

15. The enteric softgel capsule of claim 1, wherein the enteric shell composition comprises less than about 1% w/w, less than about 0.5 w/w, less than about 0.25 w/w, less than about 0.1% w/w, less than about 0.05 w/w, less than about 0.01% w/w, or 0% w/w of conventional enteric polymers, based on total weight of the shell composition.

16. The enteric softgel capsule of claim 15, wherein conventional enteric polymers comprise one or more of acid-insoluble polymers, acrylic and methacrylic acid polymers, methyl acrylate-methacrylic acid copolymers, cellulose acetate succinate, cellulose acetate phthalate, cellulose acetate butyrate, hydroxypropyl methyl cellulose phthalate, hydroxypropyl methyl cellulose acetate succinate (hypromellose acetate succinate), polyvinyl acetate phthalate (PVAP), algenic acid salts such as sodium alginate and potassium alginate, stearic acid and shellac, or mixtures thereof.

17. (canceled)

18. The enteric softgel capsule of claim 1, wherein the enteric shell composition comprises carrageenan that is at least one of iota-carrageenan, kappa-carrageenan and mixtures thereof.

19. The enteric softgel capsule of claim 1, wherein about 70% or more, about 75% or more, about 80% or more, about 85% or more, or about 90% or more of the proton pump inhibitor is dissolved in about 30 minutes at pH of about 6.8 during a buffer stage when the fill material is tested using UPLC and pursuant to USP41-NF36.

20. The enteric softgel capsule of claim 1, wherein about 90% w/w or more, about 95% w/w or more, about 98% w/w or more, about 99% w/w or more, or about 99.5% w/w or more, or about 99.8% w/w or more of the proton pump inhibitor remains in the enteric softgel capsule after being subjected to a stability test performed under stressed conditions at a temperature of about 25° C. and 60% relative humidity for about 2 weeks.

21. (canceled)

22. The enteric softgel capsule of claim 1, wherein about 90% w/w or more, about 95% w/w or more, wherein about 98% w/w or more, about 99% w/w or more, or about 99.5% w/w or more, or about 99.8% w/w or more of the proton pump inhibitor remains in the enteric softgel capsule after being subjected to a stability test performed under stressed conditions at a temperature of about 25° C. and 60% relative humidity for about 4 weeks.

23. (canceled)

24. The enteric softgel capsule of claim 1, wherein enteric shell does not rupture for at least about one hour, at least about two hours, at least about three hours, at least about four hours, or at least about five hours when the softgel capsule is in 0.1N HCl at about 37±2° C.

25.-27. (canceled)

28. The enteric softgel capsule of claim 1, wherein the fill material comprises a surfactant and/or dispersing agent that is polysorbate 80.

29. (canceled)

30. (canceled)

31. The enteric softgel capsule of claim 1, wherein the fill material comprises an alkalizing agent that is magnesium oxide.

32.-42. (canceled)

43. The enteric softgel capsule of claim 1, wherein the enteric shell composition comprises a plasticizer that is at least one of glycerin, sorbitol and mixtures thereof.

44. (canceled)

45. (canceled)

46. (canceled)

47. (canceled)

48. A process for preparing the enteric softgel capsule according to claim 1, the process comprising:
(a) preparing the fill material; and
(b) encapsulating the fill material with the enteric shell composition.

49.-58. (canceled)

59. A fill material comprising a proton pump inhibitor, about 60% w/w to about 90% w/w of a suspension system, about 0.1% w/w to about 5% w/w of an antioxidant, about 0.5% w/w to about 5% w/w of a surfactant and/or dispersing agent, and about 0.5% w/w to about 10% w/w of an alkalizing agent, based on total weight of the fill material; and
wherein about 60% or more of the proton pump inhibitor is dissolved in about 30 minutes at pH of about 6.8 during a buffer stage when the fill material is tested using UPLC and pursuant to USP41-NF36.

60. (canceled)
